(19) United States
(12) Patent Application Publication
(10) Pub. No.: US 2010/0179234 A1
(43) Pub. Date: Jul. 15, 2010

(54) TRANSITION METAL NANOCATALYST, METHOD FOR PREPARING THE SAME, AND PROCESS FOR FISCHER-TROPSCH SYNTHESIS USING THE SAME

(75) Inventors: Yuan Kou, Beijing (CN); Ning Yan, Beijing (CN); Chaoxian Xiao, Beijing (CN); Zhipeng Cai, Beijing (CN); Yongwang Li, Beijing (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Synfuels China Technology Co., Ltd., Beijing (CN)

(21) Appl. No.: 12/593,607

(22) PCT Filed: Apr. 30, 2008
(86) PCT No.: PCT/CN2008/000886

§ 371 Date: Mar. 18, 2010

(30) Foreign Application Priority Data
May 8, 2007 (CN) 200710099011.X

(51) Int. Cl. B01J 31/06; C07C 1/06

(52) U.S. Cl. 518/716; 502/159; 518/715

(57) ABSTRACT

The present invention discloses a transition metal nano-catalyst, a method for preparing the same, and a process for Fischer-Tropsch synthesis using the catalyst. The transition metal nano-catalyst comprises transition metal nanoparticles and polymer stabilizers, and the transition metal nanoparticles are dispersed in liquid media to form stable colloids. The transition metal nano-catalyst can be prepared by mixing and dispersing transition metal salts and polymer stabilizers in liquid media, and then reducing the transition metal salts with hydrogen at 100-200° C. The nano-catalyst can be used for F-T synthesis reaction. The process for F-T synthesis using the nano-catalyst comprises contacting a reactant gas mixture comprising carbon monoxide and hydrogen with the catalyst and reacting. The catalyst can rotate freely in three-dimensional space under reaction conditions, and have excellent catalystic activity at a low temperature of 100-200° C. Those reaction conditions are much milder than those for current industrial catalysts for F-T synthesis (200-350° C.). In addition, the transition metal nanoparticles have smaller diameter and narrower diameter distribution, which is beneficial to control product distribution. Meanwhile, the catalyst can be easily separated from hydrocarbon products and reused. All of the above merits imply the broad application prospects of the transition metal nano-catalyst.

### FIELD OF THE INVENTION

The present invention relates to a transition metal nano-catalyst, a method for preparing the same, and a process for Fischer-Tropsch synthesis using the above catalyst.

### BACKGROUND OF THE INVENTION

Fischer-Tropsch synthesis is a reaction that produces hydrocarbons from carbon monoxide and hydrogen (commonly known as syngas) over some metal catalysts including iron, cobalt, ruthenium etc. The products of Fischer-Tropsch synthesis have a very broad and continuous distribution starting from C₁ product (methane). With the depletion of crude oil, Fischer-Tropsch synthesis become more and more important, since it can produce hydrocarbons (i.e., gasoline and diesel fuel) from relatively abundant coal, natural gas and biomass via syngas as intermediate, thus reduces the dependence on petroleum resource, and is of great importance for both energy security and economy.

Currently, the selectivities of desired gasoline and diesel components (mainly C₅⁺ hydrocarbon) need to be improved, while the selectivity of unwanted methane need to be reduced under the typical reaction conditions for Fischer-Tropsch synthesis. Also, the conversion of carbon monoxide in a single pass is generally not high, increasing operational cost for syngas recycling. Furthermore, Fischer-Tropsch synthesis is an exothermic reaction, which favors low temperature. However, reaction temperature in current process is normally 200-350° C., a relatively high temperature that may result in catalyst sintering. In addition, bulky fused iron catalyst or iron, cobalt and ruthenium catalysts supported on silica are widely used in current process of Fischer-Tropsch synthesis. Those catalysts have rather poor catalystic activity, because of their low surface area, limited active sites, and lack of free rotation in three-dimensional space for being restricted by surface of supports. In literature, ruthenium has been reported to be the most active catalyst for Fischer-Tropsch synthesis, and then iron and cobalt. The catalystic reaction is often carried out at 200-350° C. under a total pressure of 0.1-5.0 MPa. Although a low temperature in the range of 100-140° C. has been reported for an unsupported ruthenium catalyst, a severe total pressure as high as 100 MPa is required (Robert B. Anderson, “The Fischer-Tropsch synthesis”, pp. 104-105, Academic Press, 1984), and high-molecular-weight polyethylenes are the main products (MW>10000).

### SUMMARY OF THE INVENTION

An object of the present invention is to provide a transition metal nano-catalyst, a method for preparing the same, and a process for Fischer-Tropsch synthesis using the catalyst.

The catalyst can rotate freely in three-dimensional space under reaction conditions, and have excellent catalystic activity at a low temperature of 100-200° C. Those reaction conditions are much milder than those for current industrial catalysts for F-T synthesis (200-350° C.). In addition, the transition metal nanoparticles have smaller diameter and narrower diameter distribution, which is beneficial to control product distribution. Meanwhile, the catalyst can be easily separated from hydrocarbon products and reused. All of the above merits imply the broad application prospects of the transition metal nano-catalyst.

The transition metal nano-catalyst of the present invention comprises transition metal nanoparticles, and polymer stabilizers, which are capable of stabilizing the transition metal nanoparticles, the transition metal nanoparticles and the polymer stabilizers are dispersed in a liquid media to form stable colloids.

The particle size of the transition metal nanoparticles is about 1-10 nm, preferably about 1.8±0.4 nm. The transition metal is selected from the group consisting of ruthenium, cobalt, nickel, iron and rhodium or any combination thereof.

A method of the present invention for preparing the transition metal nano-catalyst comprises the steps of mixing and dispersing transition metal salts and polymer stabilizers in a liquid media, then reducing the transition metal salts with hydrogen at about 100-200° C., to obtain the above transition metal nano-catalyst.

The reduction reaction is carried out under a total pressure of about 0.1-4.0 MPa at about 100-200° C. for about 2 hours. The molar ratio of polymer stabilizers to transition metal salts is between 400:1 to 1:1, preferably 200:1 to 1:1. The concentrations of transition metal salts dissolved in liquid media are 0.0014-0.014 mol/L. The transition metal salts are selected from salts of the following metals of a group consisting of ruthenium, cobalt, nickel, iron and rhodium or any combination thereof. The polymer stabilizers are selected from poly(N-vinyl-2-pyrrolidone) (PVP) or poly[(N-vinyl-2-pyrrolidone)-co-(1-vinyl-3-alkylimidazolium halide)] (abbreviated as [BVIMPVP]Cl prepared by a method referred to the literature: Xin-dong Mu, Jian-qiang Meng, Zi-Chen Li, and Yuan Kou, Rhodium Nanoparticles Stabilized by Ionic Copolymers in Ionic Liquids: Long Lifetime Nanocluster Catalysts for Benzene Hydrogenation, J. Am. Chem. Soc. 2005, 127, 9694-9695). The liquid media are selected from a group consisting of water, alcohols, hydrocarbons, ethers, and ionic liquids; preferably water, ethanol, cyclohexane, 1,4-dioxane, or 1-butyl-3-methylimidazolium tetrafluoroborate (abbreviated as [BMIM][BF₄]) ionic liquid.

In another aspect, the present invention relates to a process for Fischer-Tropsch synthesis using the transition metal nano-catalyst of the present invention wherein carbon monoxide and hydrogen are contacted with the catalyst and reacted for Fischer-Tropsch synthesis.

For the F-T synthesis reaction, the reaction temperature is between about 100° C.-200° C., preferably about 150° C.; the total pressure of CO and H₂ is 0.1-10 MPa, preferably about 3 MPa; the molar ratio of H₂/CO is in the range of about 0.5-3:1, preferably about 0.5, 1.0 or 2.0.

### DESCRIPTION OF FIGURES

FIG. 1 shows transmission electron micrograph and particle size distribution of ruthenium nano-catalyst of the present invention.

### DETAILED DESCRIPTION OF THE INVENTION

A method of the present invention for preparing transition metal nano-catalyst comprises the steps of mixing and dispersing transition metal salts and polymer stabilizers in a liquid media, then reducing the transition metal salts with hydrogen at the temperature of 100-200° C., to obtain the transition metal nano-catalyst.

Wherein, the transition metal salts are selected from a group consisting of RuCl₃.nH₂O, CoCl₂.6H₂O, NiCl₂.6H₂O, FeCl₃.6H₂O and RhCl₃.nH₂O or any combination thereof; while a combination of the above transition metal salts is chosen, a composite transition metal nano-catalyst can be obtained. The polymer stabilizers are selected from poly(N-vinyl-2-pyrrolidone) (PVP) or poly[(N-vinyl-2-pyrrolidone)-co-(1-vinyl-3-alkylimidazolium halide)] (abbreviated as [BVIMPVP]Cl, which is prepared by a method referred to literature: Xin-dong Mu, Jian-qiang Meng, Zi-Chen Li, and Yuan Kou, Rhodium Nanoparticles Stabilized by Ionic Copolymers in Ionic Liquids: Long Lifetime Nanocluster Catalysts for Benzene Hydrogenation, J. Am. Chem. Soc. 2005, 127, 9694-9695). The liquid media are selected from a group consisting of water, alcohols, hydrocarbons, ethers, ionic liquids and the like; preferably water, ethanol, cyclohexane, 1,4-dioxane, or [BMIM][BF₄] (1-butyl-3-methylimidazolium tetrafluoroborate) ionic liquid. The molar ratio of polymer stabilizers to transition metal salts is between 400:1-1:1, preferably 200:1-1:1. The concentrations of transition metal salts dissolved in liquid media are in the range of 0.0014-0.014 mol/L.

Preferably, for the reduction reaction the total pressure is 0.1-4.0 MPa, and more preferably 2 MPa, the reaction temperature is 150° C., and reaction time is 2 hours.

The Fischer-Tropsch synthesis reaction using the transition metal nano-catalyst comprises the steps of introducing syngas of carbon monoxide and hydrogen with an appropriate pressure in the presence of transition metal nano-catalyst, and reacting at appropriate temperature in a liquid reaction media in which the catalyst is homogenously dispersed.

In the Fischer-Tropsch synthesis reaction, the reaction temperature is between 100° C.-200° C., preferably about 150° C.; total pressure is in the range of 0.1-10 MPa, preferably about 3 MPa; molar ratio of hydrogen to carbon monoxide is between 0.5-3:1, preferably about 0.5, 1.0 or 2.0.

The products under various reaction conditions have consistent distributions and mainly comprise normal paraffin, small quantities of branched paraffin and α-olefin. For example, the typical product distribution is as follows: C₁ 3.4-6.3 wt %, C₂-C₄ 13.2-18.0 wt %, C₅-C₁₂ 53.2-56.9 wt %, C₁₃-C₂₀ 16.9-24.2 wt %, and C₂₁⁺ 1.5-4.9 wt %. It is noteworthy that desired C₅⁺ products are accounted 76.7-83.4 wt % based on total products.

The following examples are exemplary procedures for preparing transition metal nano-catalyst and carrying out process for Fischer-Tropsch synthesis using the same according to the present invention.

### Example 1

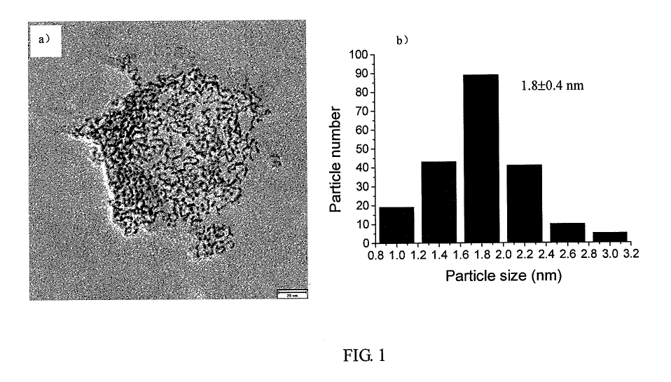

73 mg of RuCl₃.nH₂O and 0.620 g of PVP (PVP:Ru=20:1, molar ratio, the same below) were dissolved in 20 ml of water with stirring. Then the mixture solution was added into a 60 ml stainless steel autoclave, and reduced with 20 atm hydrogen at 150° C. for 2 hours to obtain the catalyst for Fischer-Tropsch synthesis in which ruthenium nanoparticles had an average diameter of 1.8±0.4 nm. Transmission electron micrograph and diameter distribution of the ruthenium nanoparticles are shown in FIGS. 1a and 1b respectively.

After cooling down to room temperature and releasing the residual gas the catalyst can be used for F-T synthesis reaction. 10 atm carbon monoxide and 20 atm hydrogen were introduced into the autoclave and reacted in 150° C. The reaction results are listed in Table 1.

### Example 2

73 mg of RuCl₃.nH₂O and 0.106 g of PVP (PVP:Ru=3.4, molar ratio) were dissolved in 20 ml of 1,4-dioxane with stirring. Then the mixture solution was added into a 60 ml stainless steel autoclave, and reduced with 20 atm hydrogen at 150° C. for 2 hours to obtained the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave, and reacted in 150° C. The reaction results are listed in Table 1.

### Example 3

73 mg of RuCl₃.nH₂O and 0.106 g of PVP (PVP:Ru=3.4, molar ratio) were dissolved in 20 ml of ethanol with stirring. Then the mixture solution was added into a 60 ml stainless steel autoclave, and reduced with 20 atm hydrogen at 150° C. for 2 hours to obtain the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave and reacted in 150° C. The reaction results are listed in Table 1.

### Example 4

73 mg of RuCl₃.nH₂O and 1.4 mmol methanol solution of poly[(N-Vinyl-2-pyrrolidone)-co-(1-vinyl-3-alkylimidazolium halide)] (abbreviated as [BVIMPVP]Cl, average monomer molecular weight 126) were dissolved in 10 ml of [BMIM][BF₄] ionic liquid with stirring. The mixture solution was heated under vacuum at 60° C. for 1 hour to remove methanol, then reduced with 20 atm H₂ at 150° C. for 2 hours in a 60 ml autoclave to obtain the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave, and reacted in 150° C. The reaction results are listed in Table 1.

### Example 5

73 mg of RuCl₃.nH₂O and 0.620 g of PVP (PVP:Ru=20, molar ratio) were dissolved in 20 ml of water with stirring. Then the mixture solution was added into a 60 ml stainless steel autoclave, and reduced with 20 atm hydrogen at 150° C. for 2 hours to obtain the catalyst for Fischer-Tropsch synthesis.

After cooling down to room temperature and releasing the residual gas the catalyst is used for F-Tsynthesis reaction. 10 atm carbon monoxide and 5 atm hydrogen were introduced into the autoclave, and reacted in 150° C. The reaction results are listed in Table 1.

### Example 6

73 mg of RuCl₃.nH₂O and 0.620 g of PVP (PVP:Ru=20, molar ratio) were dissolved in 20 ml of water with stirring. Then the mixture solution was added into a 60 ml stainless steel autoclave, and reduced with 20 atm hydrogen at 150° C. for 2 hours to obtain the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave and reacted in 100° C. The reaction results are listed in Table 1.

### Example 7

73 mg of RuCl₃.nH₂O and 0.062 g of PVP (PVP:Ru=20, molar ratio) were dissolved in 20 ml of water with stirring. Then the mixture solution was added into a 60 ml stainless steel autoclave, and reduced with 20 atm hydrogen at 150° C. for 2 hours to obtain the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave and reacted in 150° C. The reaction results are listed in Table 1.

### Example 8

73 mg of RuCl₃.nH₂O and 6.20 g of PVP (PVP:Ru=200, molar ratio) were dissolved in 20 ml of water with stirring. Then the mixture solution was added into a 60 ml stainless steel autoclave, and reduced with 20 atm hydrogen at 150° C. for 2 hours to obtain the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave and reacted in 150° C. The reaction results are listed in Table 1.

### Example 9

119 mg of CoCl₂.6H₂O and 2.25 g of PVP (PVP:Co=40, molar ratio) were dissolved in 50 ml of water with stirring. Then the mixture solution was added into a 100 ml stainless steel autoclave, and reduced with 40 atm hydrogen at 170° C. for 2 hours to obtain the catalyst for Fischer-Tropsch synthesis.

After cooling down to room temperature and releasing the residual gas the catalyst is used for F-Tsynthesis reaction. 10 atm carbon monoxide and 20 atm hydrogen were introduced into the autoclave and reacted in 170° C. The reaction results are listed in Table 1.

### Example 10

136 mg of FeCl₃.6H₂O and 5.63 g of PVP (PVP:Co=100, molar ratio) were dissolved in 50 ml of water with stirring. Then the mixture solution was added into a 100 ml stainless steel autoclave, and reduced with 40 atm hydrogen at 200° C. for 2 hours to obtain the catalyst for Fischer-Tropsch synthesis.

After cooling down to room temperature and releasing the residual gas the catalyst is used for F-Tsynthesis reaction. 20 atm carbon monoxide and 40 atm hydrogen were introduced into the autoclave and reacted in 200° C. The reaction results are listed in Table 1.

TABLE 1
Catalytic activity of the transition metal nanoparticles in various solvents for Fischer-Tropsch synthesis

| | | Decrease of | | Turnover frequency* |
|---|---|---|---|---|
| Examples | Reaction conditions | total pressure | | (mol_{CO}/mol_{Ru}⁻h) |
| Exp. 1 | PVP:Ru = 20:1, 20.0 ml water, 2.79 × 10⁻⁴ mol Ru, | 26.2 | atm/14 h | 6.1 |
| | 150° C., 20.0 atm H₂, 10.0 atm CO | | | |
| Exp. 2 | PVP:Ru = 3.4:1, 20.0 ml 1,4-dioxane, | 1 | atm/8 h | 0.42 |
| | 2.79 × 10⁻⁴ mol Ru, 150° C., 20.0 atmH₂, 10.0 atmCO | | | |
| Exp. 3 | PVP:Ru = 3.4:1, 20.0 ml ethanol, 2.79 × 10⁻⁴ mol Ru, | 1 | atm/10 h | 0.32 |
| | 150° C., 20.0 atmH₂, 10.0 atmCO | | | |
| Exp. 4 | [BVIMPVP]Cl:Ru = 5:1, 10.0 ml[BMIM][BF₄] | 3.2 | atm/14.3 h | 0.52 |
| | ionic liquid, 2.79 × 10⁻⁴ mol Ru, 150° C., | | | |
| | 20.0 atm H₂, 10.0 atm CO | | | |
| Exp. 5 | PVP:Ru = 20:1, 20.0 ml water, 2.79 × 10⁻⁴ mol Ru, | 8 | atm/11.5 h | 2.3 |
| | 150° C., 5.0 atm H₂, 10.0 atm CO | | | |
| Exp. 6 | PVP:Ru = 20:1, 20.0 ml water, 2.79 × 10⁻⁴ mol Ru, | 3.4 | atm/15 h | 0.74 |
| | 100° C., 20.0 atm H₂, 10.0 atm CO | | | |
| Exp. 7 | PVP:Ru = 20:1, 20.0 ml water, 2.79 × 10⁻⁵ mol Ru, | 6.2 | atm/15.5 h | 13 |
| | 150° C., 20.0 atm H₂, 10.0 atm CO | | | |
| Exp. 8 | PVP:Ru = 200:1, 20.0 ml water, 2.79 × 10⁻⁴ mol Ru, | 22.5 | atm/20.7 h | 3.54 |
| | 150° C., 20.0 atm H₂, 10.0 atm CO | | | |
| Exp. 9 | PVP:Co = 40:1, 50.0 ml water, 5.0 × 10⁻⁴ mol Co, | 0.2 | atm/24 h | 0.020 |
| | 170° C., 20.0 atm H₂, 10.0 atm CO | | | |
| Exp. 10 | PVP:Fe = 100:1, 50.0 ml water, 5.0 × 10⁻⁴ mol Fe, | 0.2 | atm/50 h | 0.0096 |
| | 200° C., 40.0 atm H₂, 20.0 atm CO | | | |

*based on CO

In Table 1, decrease of total pressure during reaction time is defined as the changes of total pressure after the reaction at room temperature; Turnover frequency is defined as moles of converted carbon monoxide per mole of metal catalyst per hour during the reaction.

The results show that transition metal nano-catalyst of the present invention has excellent catalystic activities at 100-150° C. The reaction temperature is remarkably lower than that for industrial Fischer-Tropsch catalysts (200-350° C.), and usable content of C₅⁺ is as high as 76.7-83.4 wt % based on the total products. The results show the bright prospects of the transition metal nano-catalyst for industrial application.

### INDUSTRIAL APPLICATIONS

A transition metal nano-catalyst is prepared in the present invention. The catalyst comprises nanoscale metal particles (1-10 nm), which can be dispersed in liquid media uniformly to form stable colloids, and the colloids do not aggregate before and after reaction. The catalyst can rotate freely in three-dimensional space under F-T synthesis reaction conditions, and have excellent catalystic activity at a low temperature of 100-200° C. Those reaction conditions are much milder than the typical F-T synthesis reaction temperature (200-350° C.) for current industrial uses. In addition, transition metal nanoparticles have smaller particle size and narrower diameter distribution than known catalysts, which is beneficial to control product distribution. Meanwhile, the catalyst can be easily separated from hydrocarbon products and can be reused. All of the above merits imply the broad application prospects of transition metal nano-catalyst of the present invention.

While the invention has been described by way of example and in terms of the specific embodiments, it is to be understood that examples and embodiments described herein are for illustrative purposes only and the invention is not limited to the disclosed embodiments. It is intended to cover various modifications and similar arrangements as would be apparent to those skilled in the art. Therefore, the scope of the appended claims should be accorded the broadest interpretation so as to encompass all such modifications and similar arrangements. All publications, patents, and patent applications cited herein are hereby incorporated by reference in their entirety for all purposes.

1. A transition metal nanocatalyst comprising transition metal nanoparticles and polymer stabilizers, wherein the transition metal nanoparticles stabilized by the polymer stabilizers are dispersed in a liquid media to form stable colloids, and particle size of the same is about 1-10 nm.

2. (canceled)

3. A transition metal nanocatalyst according to claim 1 characterized in that the particle size of said transition metal nanoparticles is about 1.8±0.4 nm.

4. A transition metal nanocatalyst according to claim 3 characterized in that the transition metal is selected from the group consisting of ruthenium, cobalt, nickel, iron and rhodium and combination thereof; the polymer stabilizers are selected from poly(N-vinyl-2-pyrrolidone) or poly[(N-vinyl-2-pyrrolidone)-co-(1-vinyl-3-alkylimidazolium halide)], and/or said liquid media is selected from the group consisting of water, alcohols, hydrocarbons, ethers and ionic liquids.

5. A transition metal nanocatalyst according to claim 4 characterized in that the liquid media is selected from water, ethanol, cyclohexane, 1,4-dioxane, or [BMIM][BF₄] ionic liquid.

6. A method for preparing the transition metal nanocatalyst according to claim 1 comprises mixing and dispersing transition metal salts and polymer stabilizers in liquid media, and reducing transition metal salts with hydrogen to obtain the transition metal nanocatalyst, wherein the temperature for the reduction reaction is about 100-200° C., and concentration of the transition metal salts dissolved in liquid media is 0.0014-0.014 mol/L.

7. A method for preparing the transition metal nanocatalyst according to claim 6 characterized in that a molar ratio of the polymer stabilizers to the transition metal salts is between 400:1 to 1:1, hydrogen pressure is 0.1-4 MPa, and the reaction time is 2 hours for the reduction reaction.

8. (canceled)

9. A method for preparing the transition metal nanocatalyst according to claim 7 characterized in that the molar ratio of the polymer stabilizers to the transition metal salts is between 200:1-1:1.

10. A method for preparing the transition metal nanocatalyst according to claim 6 characterized in that the transition metal salts are selected from the group consisting of RuCl₃.nH₂O, CoCl₂.6H₂O, NiCl₂.6H₂O, FeCl₃.6H₂O, RhCl₃.nH₂O and combinations thereof; the polymer stabilizers are selected from poly(N-vinyl-2-pyrrolidone) or poly[(N-vinyl-2-pyrrolidone)-co-(1-vinyl-3-alkylimidazolium halide)]; and/or the liquid media is selected from the group consisting of water, alcohols, hydrocarbons, ethers and ionic liquids.

11. A method for preparing the transition metal nanocatalyst according to claim 10 characterized in that the liquid media is selected from water, ethanol, cyclohexane, 1,4-dioxane, or [BMIM][BF₄] ionic liquid.

12. (canceled)

13. (canceled)

14. (canceled)

15. (canceled)

16. A process for Fischer-Tropsch synthesis characterized in that the Fischer-Tropsch synthesis reaction is performed by using transition metal nanocatalyst according to claim 1 for converting CO and H₂ into hydrocarbons.

17. A process for Fischer-Tropsch synthesis according to claim 16 characterized in that the reaction temperature for Fischer-Tropsch synthesis is 100-200° C.

18. (canceled)

19. A process for Fischer-Tropsch synthesis according to claim 16 characterized in that total pressure of carbon monoxide and hydrogen for Fischer-Tropsch synthesis is 0.1-10 MPa, and/or molar ratio of H₂ to CO is 0.5-3:1.

20. (canceled)

21. (canceled)

22. (canceled)

23. (canceled)

24. (canceled)

25. (canceled)

26. (canceled)

27. A transition metal nanocatalyst according to claim 1 characterized in that the nanocatalyst is prepared by the following processes: mixing and dispersing transition metal salts and polymer stabilizers in liquid media, and reducing transition metal salts with hydrogen at 100-200° C. to obtain the transition metal nanocatalyst.

28. A transition metal nanocatalyst according to claim 27 characterized in that the transition metal salts are selected from a group consisting of RuCl₃.nH₂O, CoCl₂.6H₂O, NiCl₂.6H₂O, FeCl₃.6H₂O, RhCl₃.nH₂O and any combination thereof.

29. A transition metal nanocatalyst according to claim 28 characterized in that hydrogen pressure is 0.1-4 MPa, reaction time is 2 hours, a molar ratio of the polymer stabilizers to the transition metal salts is between 400:1 to 1:1, and/or concentration of the transition metal salts dissolved in liquid media is 0.0014-0.014 mol/L for the reduction reaction.

30. A transition metal nanocatalyst according to claim 29 characterized in that the molar ratio of the polymer stabilizers to the transition metal salts is between 200:1 to 1:1.

31. A process of Fischer-Tropsch synthesis according to claim 17 or 19 characterized in that the reaction temperature for Fischer-Tropsch synthesis is 100° C. or 150° C., the total reaction pressure of H₂ and CO is about 3 MPa, and/or a molar ratio of H₂ to CO is about 0.5, 1.0 or 2.0.
